(19) United States
(12) Patent Application Publication
(10) Pub. No.: US 2026/0141527 A1
(43) Pub. Date: May 21, 2026

(54) APPARATUS AND METHOD FOR ENABLING QUANTITATIVE THYROID SPECT WITHOUT CT BY USING NEURAL NETWORK

(71) Applicant: SEOUL NATIONAL UNIVERSITY HOSPITAL, Seoul (KR)

(72) Inventors: Won Woo LEE, Seoul (KR); Kyounghyoun KWON, Gwangju-si, Gyeonggi-do (KR)

(21) Appl. No.: 19/099,464

(22) PCT Filed: Jun. 1, 2023
(86) PCT No.: PCT/KR2023/007541

§ 371 Date: Jan. 12, 2026

(30) Foreign Application Priority Data
Aug. 2, 2022 (KR) 10-2022-0096351
Dec. 9, 2022 (KR) 10-2022-0171471

(51) Int. Cl. G06T 7/00; A61B 6/03; A61B 6/50; G06T 7/12

(52) U.S. Cl. CPC G06T 7/0016; A61B 6/037; A61B 6/50; G06T 7/12; G06T 2207/10081; G06T 2207/10108; G06T 2207/20081; G06T 2207/20084; G06T 2207/20212; G06T 2207/30004

(57) ABSTRACT

An apparatus and a method for enabling a quantitative thyroid SPECT without CT using a neural network, according to one embodiment, are disclosed. The apparatus for enabling a quantitative thyroid SPECT without CT using a neural network, according to one embodiment, comprises: a generation unit for generating an attenuation map by inputting a first single photon emission computed tomography (SPECT) image and a second SPECT image of the thyroid into a first model; a segmentation unit for segmenting a thyroid map by inputting the attenuation map and the first SPECT image into a second model; and a calculation unit for calculating the radiopharmaceutical uptake amount of the thyroid by using the first SPECT images sinogram, the second SPECT images sinogram, the attenuation map, and the thyroid map.

### TECHNICAL FIELD

The present disclosure relates to techniques for enabling quantitative thyroid single-photon emission computed tomography (SPECT) without computed tomography (CT) using a neural network.

### BACKGROUND ART

Thyroid function is assessed by measuring the uptake amount (%) of radiopharmaceuticals administered intravenously into the thyroid in thyroid patients. Typically, the measurement of the uptake amount of radiopharmaceuticals is mainly evaluated by a thyroid uptake system, but it has the limitation of low accuracy.

Therefore, single-photon emission computed tomography/computed tomography (SPECT/CT), which has been reported to have higher accuracy in measuring the uptake amount than the thyroid uptake system, is attracting attention.

The SPECT/CT is a nuclear medicine imaging method that provides quantitative information using radiopharmaceuticals, such as Tc-99m pertechnetate. The SPECT/CT uses CT for quantitative measurements. The CT performs attenuation correction to accurately measure the uptake amount of radiopharmaceuticals into the thyroid and is used for thyroid segmentation.

However, the method for evaluating thyroid function using SPECT/CT results in a total radiation exposure of about 3.34 mSv, combining radiation exposure by CT of about 1.12 mSv with radiation exposure by SPECT of about 2.22 mSv. Furthermore, thyroid segmentation has the cumbersome disadvantage of requiring individual segmentation by a specialist.

### DETAILED DESCRIPTION OF THE INVENTION

### Technical Problem

The disclosed embodiments are intended to enable quantitative thyroid single-photon emission computed tomography (SPECT) without computed tomography (CT) using a neural network.

### Solution to Problem

A device for enabling quantitative thyroid single-photon emission computed tomography (SPECT) without computed tomography (CT) using a neural network according to an embodiment includes: a generation unit configured to generate an attenuation map by inputting a first SPECT image and a second SPECT image of the thyroid into a first model: a segmentation unit configured to segment the thyroid map by inputting the attenuation map and the first SPECT image into a second model; and a calculation unit configured to calculate an uptake amount of radiopharmaceuticals into the thyroid using a first SPECT image sinogram, a second SPECT images sinogram, the attenuation map, and the thyroid map.

The first SPECT image may be a primary emission SPECT image.

The second SPECT image may be a scattering SPECT image.

The generation unit may generate a third SPECT image by performing attenuation correction (AC) on the first SPECT image sinogram using the attenuation map, performing scatter correction (SC) on the first SPECT image sinogram using the second SPECT image sinogram, and applying resolution recovery (RR) to the first SPECT images sinogram.

The third SPECT image may be a quantitative ACSCRR SPECT image.

The calculation unit may calculate the uptake amount of radiopharmaceuticals into the thyroid by combining the third SPECT image with the thyroid map.

The calculation unit may calculate the uptake amount of radiopharmaceuticals into the thyroid by combining the third SPECT image with the thyroid map to count matching voxels.

The second model may be trained to segment the thyroid map in the input image based on a plurality of thyroid segmentation maps drawn and labeled along the outline of the thyroid in the CT image.

A method for enabling quantitative thyroid SPECT without CT using a neural network according to an embodiment is a method performed by a device for enabling quantitative thyroid SPECT without CT using a neural network having one or more processors and memory storing one or more programs to be executed by the one or more processor. The method includes: generating an attenuation map by inputting a first SPECT image and a second SPECT image for the thyroid into a first model: segmenting a thyroid map by inputting the attenuation map and the first SPECT image into a second model; and calculating an uptake amount of radiopharmaceuticals into the thyroid by using a first SPECT images sinogram, a second SPECT image sinogram, the attenuation map, and the thyroid map.

The first SPECT image may be a primary emission SPECT image.

The second SPECT image may be a scattering SPECT image.

The generating of the attenuation map may include generating a third SPECT image by performing AC on the first SPECT image sinogram using the attenuation map, performing SC on the first SPECT image sinogram using the second SPECT image sinogram, and applying RR to the first SPECT images sinogram.

The third SPECT image may be a quantitative ACSCRR SPECT image.

The calculating of the uptake amount may include calculating an uptake amount of radiopharmaceuticals into the thyroid by combining the third SPECT image with the thyroid map.

The calculating of the uptake amount may include calculating an uptake amount of radiopharmaceuticals into the thyroid by combining the third SPECT image with the thyroid map to count matching voxels.

The second model may be trained to segment the thyroid map in the input image based on a plurality of thyroid segmentation maps drawn and labeled along the outline of the thyroid in the CT image.

### Effects of the Invention

The disclosed embodiments may desirably reduce radiation exposure by computed tomography (CT) by performing attenuation correction using a neural network instead of CT, according to the As Low As Reasonably Achievable (ALARA) principle.

The disclosed embodiments may segment the thyroid map using the neural network to provide the thyroid map without human intervention.

### BRIEF DESCRIPTION OF THE DRAWINGS

FIG. 1 is a block diagram of a device for enabling quantitative thyroid single-photon emission computed tomography (SPECT) without computed tomography (CT) using a neural network according to an embodiment.

FIG. 2 is a block diagram illustrating a learning model of a device for enabling quantitative thyroid SPECT without CT using a neural network according to an embodiment.

FIGS. 3A to 4C illustrate the performance of a device for enabling quantitative thyroid SPECT without CT using a neural network according to an embodiment.

FIG. 5 is a flowchart of a method for enabling quantitative thyroid SPECT without CT using a neural network.

### BEST MODE FOR CARRYING OUT THE INVENTION

Although terms used herein are selected from general terms currently widely used while considering functions, the terms may vary according to the intention or practice of those skilled in the art or the emergence of new technologies. In addition, in a specific case, there are terms arbitrarily selected by the applicant and the meaning thereof is described in the specification. Thus, the terms used herein is to be interpreted based on the actual meaning of the terms and its context throughout the specification, rather than the mere name of the terms.

Terms, such as first or second, used herein may be used to describe various components, but the components should not be limited by the terms. Only for the purpose of distinguishing one component from another, without departing from the scope of the present inventive concept, a first component may be named a second component, and similarly, the second component may also be named the first component.

As used herein, the singular forms include plural forms unless the context clearly indicates otherwise. In this application, terms, such as “comprise,” “include,” or “have,” are intended to represent the presence of the elements or a combination thereof described in the specification but do not preclude the presence or addition of other elements or features.

In addition, embodiments described herein may be implemented entirely in hardware, partly in hardware and partly in software, or entirely in software. In this specification, a “unit,” a “module,” a “device,” a “server,” or a “system” refers to a computer-related entity, such as hardware, a combination of hardware and software, or software. For example, the unit, the module, the device, the server, or the system may refer to hardware constituting a part or all of a platform and/or software, such as an application for driving the hardware.

Hereinafter, embodiments are described in detail with reference to the accompanying drawings and the contents described in the accompanying drawings. However, the scope to be claimed is not limited by the embodiments.

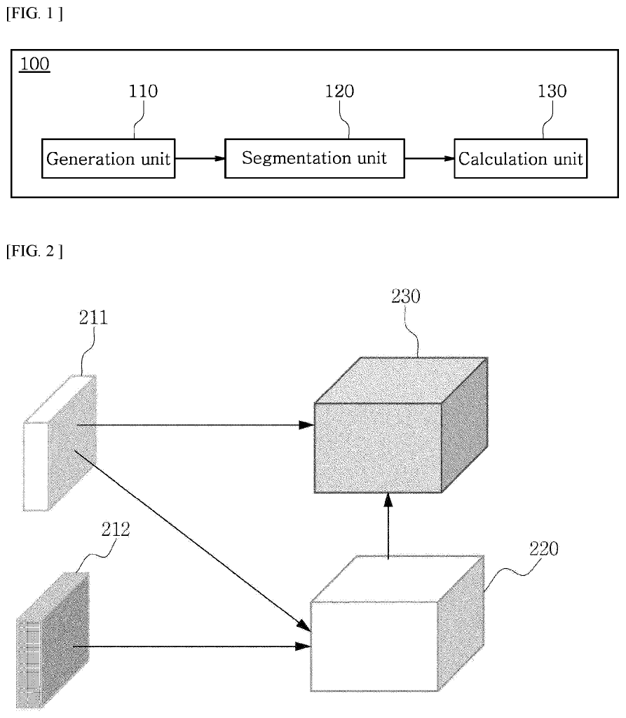

FIG. 1 is a block diagram of a device 100 for enabling quantitative thyroid single-photon emission computed tomography (SPECT) without computed tomography (CT) using a neural network according to an embodiment.

Referring to FIG. 1, the device 100 for enabling quantitative thyroid SPECT without CT using a neural network includes a generation unit 110, a segmentation unit 120, and a calculation unit 130.

The generation unit 110, the segmentation unit 120, and the calculation unit 130 may be implemented by using one or more physically separated devices or may be implemented by one or more processors or a combination of the one or more processors and software. Alternatively, the generation unit 110, the segmentation unit 120, and the calculation unit 130 may not be clearly distinguished in specific operations, unlike the illustrated examples.

The generation unit 110 generates an attenuation map by inputting a first SPECT image and a second SPECT image of the thyroid into a first model.

The SPECT is a nuclear medicine imaging technique for reconstructing the distribution of single photon emitting nuclides in the body into an image by injecting radiopharmaceuticals emitting single photons (gamma rays) into the body, and then measuring the transmitted gamma rays in the body. In particular, the SPECT may provide a bio-functional image, which is a biochemical phenomenon of the body.

The first SPECT image may be a primary emission SPECT image generated using the SPECT. The primary emission SPECT image may be a SPECT image for primary gamma rays having the highest frequency in the SPECT.

The second SPECT image may be a scattering SPECT image generated using the SPECT. The scattering SPECT image may be an SPECT image of gamma rays scattered during the SPECT.

At least one of the first SPECT image and the second SPECT image may be an NCRR SPECT image corrected for a collimator-detector response, i.e., resolution recovery, for improved accuracy. Specifically, at least one of the first SPECT image and the second SPECT image may be an image corrected for resolution recovery using a Butterworth low-pass filter for statistical noise reduction.

At least one of the first SPECT image and the second SPECT image may be an image normalized to a maximum value of a composite image of the two SPECTs.

The generation unit 110 may generate a third SPECT image by performing attenuation correction (AC) on the first SPECT image sinogram by using the attenuation map.

The generation unit 110 may generate the third SPECT image by performing at least one of AC, scatter correction (SC), and resolution recovery (RR) by using the attenuation map.

In this case, the third SPECT image may be a quantitative ACSCRR SPECT image. The quantitative ACSCRR SPECT image may be a SPECT image in which the SPECT image sinogram for primary gamma rays is corrected.

The segmentation unit 120 inputs the attenuation map and the first SPECT image to a second model to segment a thyroid map.

The calculation unit 130 calculates the uptake amount of radiopharmaceuticals into the thyroid using a first SPECT image sinogram, a second SPECT image sinogram, the attenuation map, and the thyroid map.

The calculation unit 130 may calculate the uptake amount of radiopharmaceuticals into the thyroid by combining the third SPECT image with the thyroid map.

The calculation unit 130 may calculate the uptake amount of radiopharmaceuticals into the thyroid by combining the third SPECT image with the thyroid map to count matching voxels.

The calculation unit 130 may calculate the uptake amount of radiopharmaceuticals into the thyroid by counting matching voxels between the third SPECT image and the thyroid map, assuming that the negative voxel value is 0.

FIG. 2 is a block diagram illustrating a learning model of the device 100 for enabling quantitative thyroid SPECT without CT by using a neural network according to an embodiment.

Referring to FIG. 2, a first SPECT image 211 and a second SPECT image 212 are input to a first model 220. The first SPECT image 211 and the attenuation map output by the first model 220 are input to a second model 230.

Specifically, when the first SPECT image 211 and the second SPECT image 212 are input by using one or more neural networks, the first model 220 may output, without CT, the attenuation map provided by the CT as an output value.

The first model 220 may include a loss function based on Equation 1 below.

$$L ( G ( X ) , Y ) = {L}_{error} ( G ( X ) , Y ) + {L}_{GDL} ( G ( X ) , Y ) \qquad [ Equation 1 ]$$

The loss function may be L (G(X),Y); Y may be a ground truth value of the attenuation map: X may be an input SPECT: G(X) may be a generated attenuation map; and Lₑᵣᵣₒᵣ may be a predefined first loss function or a second loss function. The L_{GDL} may be a gradient difference loss (GDL) for sharpness of the generated attenuation map.

For example, the first loss function may be a function that calculates a sum of differences in absolute values between the ground truth value of the attenuation map and the generated attenuation map. In other words, the first loss function L1 may be set based on Equation 2 below.

$$L 1 = Σ \vert G ( X ) - Y \vert \qquad [ Equation 2 ]$$

For example, the second loss function may be a function that calculates a sum of squares of differences between the ground truth value of the attenuation map and the generated attenuation map. In other words, the second loss function L2 may be set based on Equation 3 below.

$$L 2 = {Σ ( G ( X ) - Y )}^{2} \qquad [ Equation 3 ]$$

For example, the L_{GDL} may be used to compensate for the imaging blur due to the loss effect of the second loss function, and the L_{GDL} may be set based on Equation 4 below.

$${L}_{GDL} = {Σ ( \vert {▽G ( X )}_{x , y , z} \vert - \vert {▽Y}_{x , y , z} \vert )}^{2} \qquad [ Equation 4 ]$$

The first model 220 may be trained based on a hyperfunction attenuation map obtained by CT scanning the thyroid determined to have hyperfunction, a hypofunction attenuation map obtained by CT scanning the thyroid determined to have hypofunction, and a standard function attenuation map obtained by CT scanning the thyroid determined to have a standard function.

The CT may scan the entire thyroid so as to cover all of the axis field of view of the SPECT or to cover only one-half to two-thirds of the axis field of view of the SPECT, thereby reducing unnecessary radiation exposure by the CT. The SPECT and CT may preferably scan from the mid-skull to the upper-mediastinum at the same axis field of view.

The CT image or the SPECT image may be labeled with a clinical diagnosis by a nuclear medicine specialist, for example, a diagnostic result for at least one of Graves' disease/hyperthyroidism, painless/subacute thyroiditis, single nodular goiter (SNG)/multinodular goiter (MNG), drug-induced thyroiditis, and lingual thyroid.

The second model 230 may be designed to segment a thyroid map as an output value when the first SPECT image 211 and the attenuation map output by the first model 220 are input by using one or more neural networks.

The second model 230 may include a loss function based on categorical cross entropy (CCE).

Specifically, the second model 230 may include a loss function based on Equation 5 below.

$$CCE = \frac{1}{n} {\sum }_{i = 1}^{n} {y}_{i} bg ( {y}_{h} ) - ( 1 - {y}_{i} ) bg ( 1 - {y}_{h} ) \qquad [ Equation 5 ]$$

yᵢ may be a ground truth value, yₕ may be a synthetic value, and n may be the number of classes of the second model 230.

For example, yᵢ may be a ground truth value represented by 0 or 1, yₕ is a synthetic value between 0 and 1, and n may be set to three: a background, a left thyroid, and a right thyroid.

In this case, the second model 230 may be trained to segment the thyroid map in the input image based on a plurality of thyroid maps drawn and labeled along the outline of the thyroid in the CT image.

At least one of the first model 220 and the second model 230 may have a U-net structure, and preferably, a 3D U-net-based structure. In other words, the first model 220 and the second model 230 may be designed to have a structure that skip-connects a contraction path to a segmentation path.

In a specific example, at least one of the first model 220 and the second model 230 may be designed to include 64 initial neurons and four skip connections. At least one of the first model 220 and the second model 230 may be a neural network based on the U-net structure that skip-connects the contraction path including one or more convolutional blocks including a 3×3 convolution layer, batch normalization, and a ReLU function to the segmentation path including the one or more convolution blocks.

In this case, max pooling, preferably 2×2×2 stride max pooling, may be applied to the convolution blocks in the contraction path, and transposed convolution, preferably 2×2×2 upconvolution, may be applied to the convolution blocks in the segmentation path.

At least one of the first model 220 and the second model 230 may further include a 1×1×1 convolution layer as a last output layer.

The first model 220 and the second model 230 may be designed to be trained end-to-end.

In the medical field, the first model 220 and the second model 230 are described to have U-net structures frequently used, which is only an example. The first model 220 and the second model 230 are designed based on various known structures, such as Seg-NET, MFU-net, generative adversarial network (GAN), and the like, and are not necessarily limited to the U-net structure.

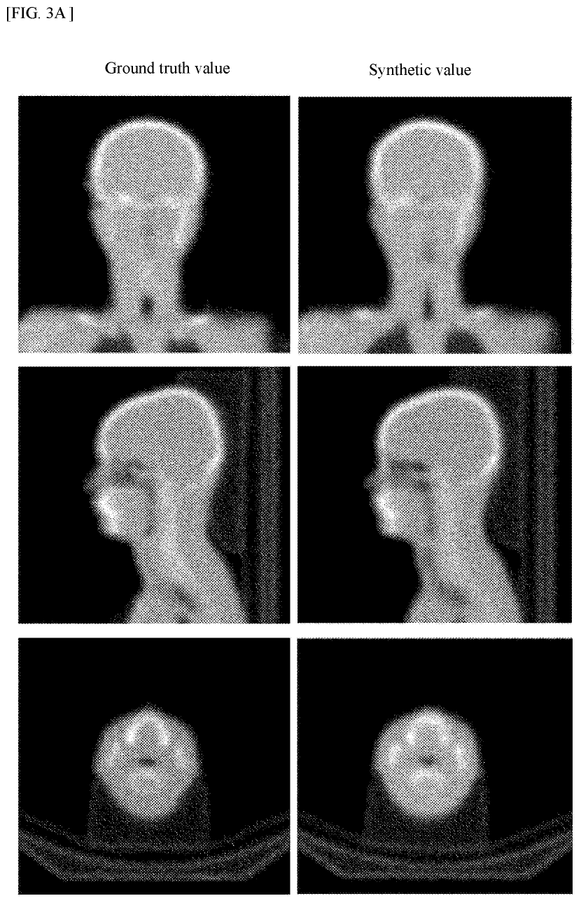

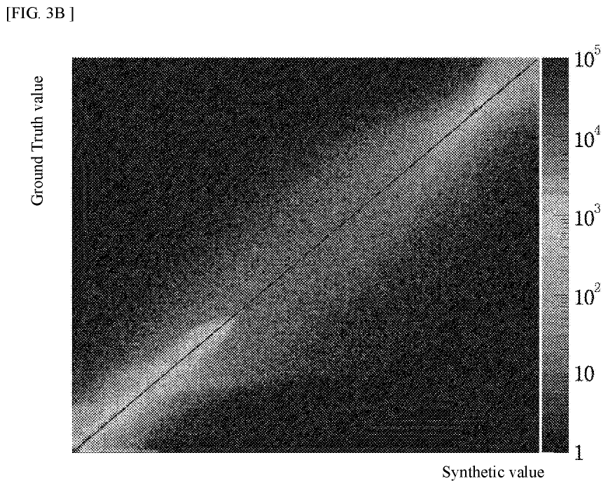

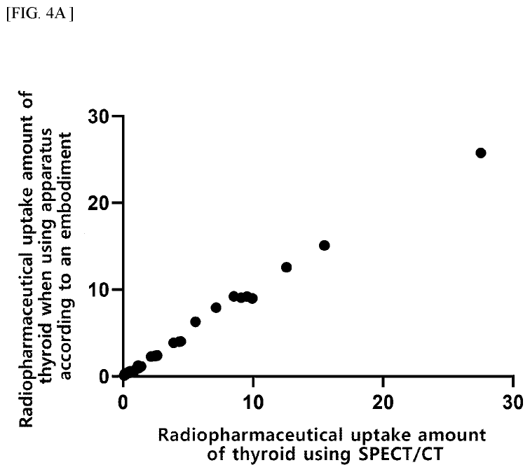

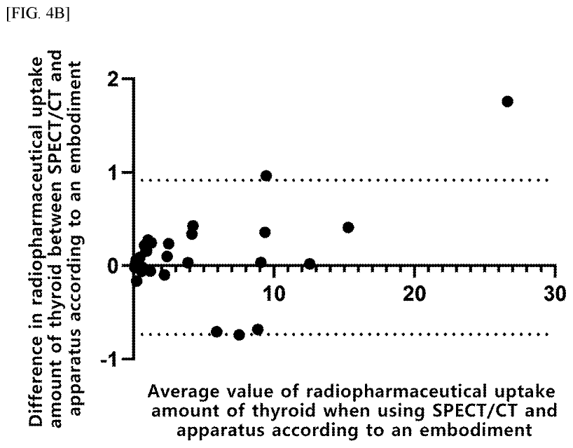

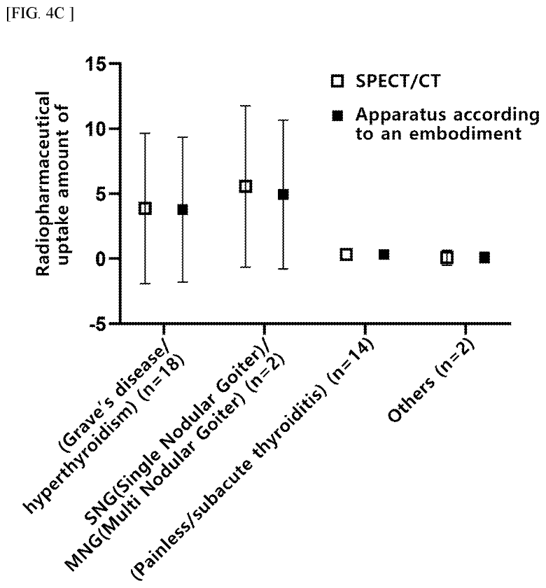

FIGS. 3A to 4C illustrate the performance of a learning model of a device for enabling quantitative thyroid SPECT without CT using a neural network according to an embodiment.

FIG. 3A is a diagram comparing a ground truth value with a synthetic value for an attenuation map.

The ground truth value on the left is an attenuation map generated through CT imaging, and the synthetic value on the right is an attenuation map generated by the device 100 for enabling quantitative thyroid SPECT without CT according to an embodiment.

FIG. 3B shows the correlation of the attenuation coefficient between the ground truth value of the attenuation map and the synthetic value of the attenuation map in FIG. 3A. FIG. 3B shows a high correlation between the ground truth value of the attenuation map and the synthetic value of the attenuation map.

FIG. 4A illustrates an uptake amount of radiopharmaceuticals into the thyroid measured by the device 100 for enabling quantitative thyroid SPECT without CT according to an embodiment and an uptake amount of radiopharmaceuticals into the thyroid measured by using the existing SPECT/CT.

In FIG. 4A, the horizontal axis represents the uptake amount of radiopharmaceuticals into the thyroid measured by SPECT/CT, and the vertical axis represents the uptake amount of radiopharmaceuticals into the thyroid measured by the device 100 for enabling quantitative thyroid SPECT without CT according to an embodiment.

The correlation between the uptake amount of radiopharmaceuticals into the thyroid measured by the existing SPECT/CT and the uptake amount of radiopharmaceuticals into the thyroid output by the device 100 for enabling quantitative thyroid SPECT without CT according to an embodiment is indicated as r=0.9980, R\hat{ }2=0.9959, p<0.0001, in the uptake range of 0 to 30. In this case, r represents a correlation coefficient in the Pearson correlation, and p represents a significance level.

FIG. 4B is a Bland-Altman plot showing the difference between the uptake amount of radiopharmaceuticals into the thyroid measured by SPECT/CT and the uptake amount of radiopharmaceuticals into the thyroid output by the device 100 for enabling quantitative thyroid SPECT without CT according to an embodiment.

The horizontal axis of FIG. 4B indicates an average value between the uptake amount of radiopharmaceuticals into the thyroid measured by the SPECT/CT and the uptake amount of radiopharmaceuticals into the thyroid output by the device 100 for enabling quantitative thyroid SPECT without the CT according to an embodiment. The vertical axis represents the difference between the uptake amount of radiopharmaceuticals into the thyroid measured by SPECT/CT and the uptake amount of radiopharmaceuticals into the thyroid output by the device 100 for enabling quantitative thyroid SPECT without CT according to an embodiment.

The Bland-Altman plot of FIG. 4B shows a non-significant systematic deviation with a bias of −0.99% to indicate the high accuracy of the device 100 for enabling quantitative thyroid SPECT without CT according to an embodiment.

FIG. 4C is a graph showing the uptake amount of radiopharmaceuticals into the thyroid output by the device 100 for enabling quantitative thyroid SPECT without CT according to an embodiment and the uptake amount of radiopharmaceuticals into the thyroid measured by SPECT/CT for each thyroid disease.

The uptake amount of radiopharmaceuticals into the thyroid output by the device 100 for enabling quantitative thyroid SPECT without CT according to an embodiment and the uptake amount of radiopharmaceuticals into the thyroid measured by SPECT/CT for each thyroid disease are shown as a mean±standard deviation value.

The thyroid diseases may be distinguished according to the thyroid absorption rate. Referring to FIG. 4B, the uptake amount of radiopharmaceuticals into the thyroid output by the device 100 for enabling quantitative thyroid SPECT without CT according to an embodiment and the uptake amount of radiopharmaceuticals into the thyroid measured by SPECT/CT show an uptake amount of radiopharmaceuticals into the thyroid for Graves' disease/hyperthyroidism, painless/subacute thyroiditis, SNG/MNG, and others (e.g., drug-induced thyroiditis, lingual thyroid).

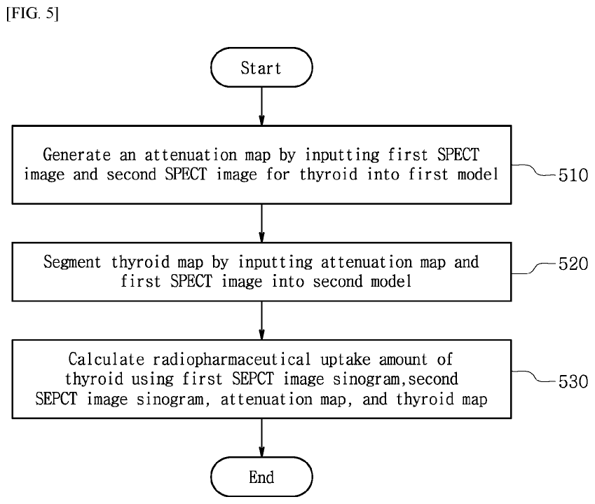

FIG. 5 is a flowchart of a method for enabling quantitative thyroid SPECT without CT using a neural network according to an embodiment.

Referring to FIG. 5, the method for enabling quantitative thyroid SPECT without CT by using a neural network according to an embodiment may be performed by the device for enabling quantitative thyroid SPECT without CT by using a neural network of FIG. 1.

First, the device for enabling quantitative thyroid SPECT without CT by using a neural network generates an attenuation map by inputting a first SPECT image and a second SPECT image of the thyroid into a first model (510).

Then, the device for enabling quantitative thyroid SPECT without CT by using a neural network segments the thyroid map by inputting the attenuation map and the first SPECT image to a second model (520).

Then, the device for enabling quantitative thyroid SPECT without CT by using a neural network calculates an uptake amount of radiopharmaceuticals into the thyroid using the first SPECT image sinogram, the second SPECT sinogram, the attenuation map, and the thyroid map (530).

The method of FIG. 5 has been described with reference to the flowchart presented in the drawing. Although, for convenience of explanation, the method is shown and described as a series of blocks, the present invention is not limited by the order of the blocks. Some blocks and the other blocks may occur simultaneously or in a different order than shown and described herein. Various other branches, flow paths, and orders of blocks may be implemented to achieve the same or similar results. Moreover, not all blocks illustrated herein may be required for implementing the method.

Furthermore, the method according to an embodiment of the present invention may be implemented in the form of a computer program for performing a series of processes, wherein the computer program may be recorded on a computer-readable recording medium. Examples of the computer-readable recording medium include magnetic media, such as a hard disk, a floppy disk, and a magnetic tape, optical recording media, such as a CD-ROM and a DVD, magneto-optical media, such as a floptical disk, and hardware devices specialized in storing and performing program instructions, such as ROM, RAM, and flash memory.

While the foregoing has been described with reference to embodiments, it will be understood by those skilled in the art that various modifications and variations can be made to the invention without departing from the spirit and scope of the invention as set forth in the following claims.

### INDUSTRIAL AVAILABILITY

The device and the method for enabling quantitative thyroid SPECT without CT using a neural network according to an embodiment is applicable to the medical diagnostic industry in the field of nuclear medicine by performing AC using the neural network instead of CT.

1. An apparatus for enabling quantitative thyroid single photon emission computed tomography (SPECT) without computed tomography (CT) using a neural network, the apparatus comprising:
a generation unit configured to generate an attenuation map by inputting a first SPECT image and a second SPECT image of the thyroid into a first model;
a segmentation unit configured to segment a thyroid map by inputting the attenuation map and the first SPECT image to a second model; and
a calculation unit configured to calculate an uptake amount of radiopharmaceuticals into the thyroid using a first SPECT image sinogram, a second SPECT image sinogram, the attenuation map, and the thyroid map.

2. The apparatus of claim 1, wherein the first SPECT image is a primary emission SPECT image, and the second SPECT image is a scattering SPECT image.

3. The apparatus of claim 1, wherein the generation unit generates a third SPECT image by performing attenuation correction (AC) on the first SPECT image sinogram using the attenuation map, performing scatter correction (SC) on the first SPECT image sinogram using the second SPECT image sinogram, and applying resolution recovery (RR) to the first SPECT images sinogram.

4. The apparatus of claim 3, wherein the third SPECT image is a quantitative ACSCRR SPECT image.

5. The apparatus of claim 3, wherein the calculation unit calculates an uptake amount of radiopharmaceuticals into the thyroid by combining the third SPECT image with the thyroid map.

6. The apparatus of claim 5, wherein the calculation unit calculates the uptake amount of radiopharmaceuticals into the thyroid by combining the third SPECT image with the thyroid map to count matching voxels.

7. The apparatus of claim 1, wherein the second model is trained to segment the thyroid map in an input image based on a plurality of thyroid segmentation maps drawn and labeled along an outline of the thyroid in a CT image.

8. A method performed by an apparatus for enabling quantitative thyroid single photon emission computed tomography (SPECT) without computed tomography (CT) using a neural network having one or more processors and memory storing one or more programs to be executed by the one or more processors, the method comprising:
generating an attenuation map by inputting a first SPECT image and a second SPECT image of the thyroid into a first model; segmenting a thyroid map by inputting the attenuation map and the first SPECT image into a second model; and calculating an uptake amount of radiopharmaceuticals into the thyroid by using a first SPECT image sinogram, a second SPECT image sinogram, the attenuation map, and the thyroid map.

9. The method of claim 8, wherein the first SPECT image is a primary emission SPECT image, and the second SPECT image is a scattering SPECT image.

10. The method of claim 8, wherein the generating of the attenuation map comprises generating a third SPECT image by performing attenuation correction (AC) on the first SPECT image sinogram using the attenuation map, performing scatter correction (SC) on the first SPECT image sinogram using the second SPECT image sinogram, and applying resolution recovery (RR) to the first SPECT images sinogram.

11. The method of claim 10, wherein the third SPECT image is a quantitative ACSCRR SPECT image.

12. The method of claim 11, wherein the calculating of the uptake amount comprises calculating an uptake amount of radiopharmaceuticals into the thyroid by combining the third SPECT image with the thyroid map.

13. The method of claim 12, wherein the calculating of the uptake amount comprises calculating an uptake amount of radiopharmaceuticals into the thyroid by combining the third SPECT image with the thyroid map to count matching voxels.

14. The method of claim 8, wherein the second model is trained to segment the thyroid map in an input image based on a plurality of thyroid segmentation maps drawn and labeled along an outline of the thyroid in a CT image.
